(19) United States
(12) Patent Application Publication
(10) Pub. No.: US 2017/0241975 A1
(43) Pub. Date: Aug. 24, 2017

(54) TEST STRIPS FOR DETERMINING THE CONCENTRATION OF A COMPONENT IN A LIQUID COMPOSITION

(71) Applicant: FULLSPEED TECHNOLOGY INC., Burnaby (CA)

(72) Inventors: Danielle T. Wilson, Burnaby (CA); Dongho Kim, Surrey (CA); Neil Branda, North Vancouver (CA); Byron D. Gates, Burnaby (CA); Nathanael Sieb, Port Coquitlam (CA); Gurdeep Ahira, Burnaby (CA); Robert Francis Boyle, JR., Madison, WI (US); Joseph Richard Wedderspoon, Verona, WI (US)

(21) Appl. No.: 15/435,316

(22) Filed: Feb. 17, 2017

Related U.S. Application Data
(60) Provisional application No. 62/298,135, filed on Feb. 22, 2016.

(51) Int. Cl. G01N 33/28; G01N 30/93; B01D 15/20; G01N 21/78

(52) U.S. Cl. CPC G01N 33/2852; G01N 21/78; G01N 30/93; B01D 15/206; G01N 2021/7759

(57) ABSTRACT

A test strip for determining the concentration of a component in a liquid composition is disclosed. The test strip comprises a carrier sheet coated with a layer of an adsorbent, and a chemical dye deposited on the test strip near but spaced apart from a bottom edge of the test strip. In some embodiments, the test strip is a thin-layer chromatography (TLC) plate, and the retention factor of the chemical dye varies according to the concentration of the component in the liquid composition. A user can determine the concentration of the component based on the migration distance of the chemical dye on the test strip in a chromatography process. The test strip can be used to perform a fast and visual test to determine the ethanol content in a fuel sample.

### TECHNICAL FIELD

The present invention relates in general to test strips for determining the concentration of a component (e.g., ethanol) in a liquid composition (e.g., a fuel mixture). The present invention also relates to methods of making the test strips and methods of using the test strips to determine the concentration of a component in a liquid composition. The present invention also relates to a test kit for determining the concentration of a component (e.g., ethanol) in a liquid composition (e.g., a fuel mixture).

### BACKGROUND

The use of alcohol-containing fuel mixtures as an energy source for engines (such as automobile engines, outdoor power equipment, small engines, etc.) has become increasingly common. Alcohols that may be blended in fuel mixtures include methanol, ethanol, propanol, butanol, and some other alcohol.

Ethanol-containing fuel mixtures have received a lot of attention, because ethanol is a renewable energy source, and can be produced from renewable sources such as sugar cane, potato, and corn.

When using ethanol-containing fuel mixtures as an energy source for automobile engines or outdoor power equipment or small engines, it is important to know the ethanol content in the fuel mixtures. Certain engines require that the ethanol content in the fuel mixture to be no more than a certain percentage (e.g., 10%), as ethanol content over a certain percentage can be harmful to the engine or have negative effects on the engine's safety and performance. This is because ethanol is corrosive and has different combustion characteristics from petroleum fuel. Additionally, some engines (especially older engines) must be modified in order to accommodate fuel mixtures that contain ethanol over a certain percentage.

Conventional chemical tests for analyzing the alcohol composition of petroleum fuel mixtures are tedious, time-consuming, and relatively expensive, and they are not fast and visual tests that can be provided in a test strip format and are not suitable for field tests.

A conventional method for determining the percentage of ethanol in gasoline involves adding a known amount of water and a known amount of sample gasoline in a graduated cylinder, plugging a stopper into the top of the graduated cylinder, and vigorously shaking the mixture in the graduated cylinder before letting the mixture to settle until phase separation occurs. Because ethanol can dissolve in water and separate from gasoline, a person can calculate the ethanol content in the gasoline by reading the new water line in the graduated cylinder. This method is not ideal, because it requires water which may not be available in the field, and second it requires about at least 20-100 ml of sample gasoline, which is relatively wasteful. It also creates a disposal issue, as the gasoline mixed with water is rendered unusable.

Therefore, it is desirable to provide test strips for rapidly and visually determining the concentration of a component (e.g., ethanol) in a fuel mixture. Such test strip-based field tests would be useful to consumers who want to quickly determine the presence or content of ethanol in a fuel mixture.

### BRIEF DESCRIPTION OF DRAWINGS

In drawings which show non-limiting embodiments of the invention:

FIG. 1 shows a test strip according to an example embodiment of the invention.

FIG. 2 shows that the example test strip can differentiate between fuel samples containing 5%, 10% and 20% ethanol and the observed retention factor of the colored chemical dye with respect to each sample is generally reproducible.

FIG. 3 shows that the example test strip can differentiate between fuel samples containing 10%, 30% and 50% ethanol.

FIG. 4 shows that the example test strip can differentiate between fuel/oil samples containing 5%, 10% and 20% ethanol.

FIG. 5 illustrates the marking of the 5%, 10% and 20% lines and the top and bottom baselines on the example test strip.

FIG. 6 shows a vial containing an example porphyrin solution and the chemical structure of 5-(4-hydroxyphenyl)-10,15,20-triphenyl porphine.

FIG. 7 shows an example test strip which has been spotted with a porphyrin solution along a bottom baseline.

FIGS. 8 and 9 show a method of using an example test strip to determine the ethanol content of a sample.

FIG. 10 shows an example of a test strip print design.

FIG. 11 shows an example test result label which can be provided as part of a test kit.

FIG. 12 is a pictorial that illustrates the steps of using a test kit to determine the ethanol content in a fuel sample.

FIG. 13A through FIG. 13E show calibration test results of example test strips using a series of fuel samples having 0%, 10%, 20%, 30% and 60% ethanol.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

In this disclosure, test strips and methods for determining the presence or content of ethanol in a liquid composition have been described; however, the invention is not limited to determining the presence or content of ethanol in a liquid composition. The invention has broader applications for determining the presence or content of other alcohols (such as methanol) or non-alcohol component in a liquid composition.

One aspect of the invention relates to test strips for determining the concentration of a component in a liquid composition. In some embodiments, the liquid composition is a blended alcohol and fuel composition. In some embodiments, the liquid composition is a blended alcohol, fuel and oil composition. The fuel can be a petroleum fuel, such as gasoline. The liquid composition may contain an alcohol, such as methanol, ethanol, propanol, or butanol. The content of the alcohol in the liquid composition can be in the range of 0-99%. For example, the test strips can determine whether ethanol is present in the sample, or whether the sample is free of ethanol (i.e., 0% ethanol), or whether the ethanol content in the sample is below or above a threshold (e.g., 5%, 10%, 20%, 30%, or 60%). In some embodiments, the test strips can rapidly and visually determine the alcohol concentration in the fuel mixture or fuel and oil mixture.

In some embodiments, the test strip comprises a thin-layer chromatography (TLC) plate. The TLC plate comprises a carrier sheet coated with a layer of an adsorbent. The carrier sheet may be glass, aluminum foil, plastic or some other suitable material. The adsorbent may be silica gel, aluminum oxide, cellulose, or some other suitable adsorbent. In some particular embodiments, the test strip may be prepared from a silica TLC plate that is commercially available (e.g., item no. 105554 from Merck KGaA). The TLC plate may be cut into a strip. For example, the strip may have a dimension of 5 cm in length and 1 cm in width, or some other suitable dimension. The strip also comprises a chemical dye that is deposited on the strip. The chemical dye should be deposited at a location near but spaced apart from the bottom edge of the strip. The chemical dye should be deposited along a line perpendicular to the longitudinal direction of the test strip.

The inventors have chosen thin-layer chromatography (TLC) instead of conventional paper chromatography or filter paper for the test strip applications. Through research and experimentation, the inventors have determined that thin-layer chromatography (TLC) provide more satisfactory results.

In use, the bottom edge of the strip is immersed into a sample (e.g., an ethanol/fuel mixture or ethanol/fuel/oil mixture). During chromatography, the silica gel of the strip serves as a stationary phase, whereas the sample mixture serves as a mobile phase. The distance the chemical dye travels up the strip is dependent on the ethanol concentration in the sample mixture. Therefore, a user can visually see the migration front of the chemical dye and determine the ethanol content of the sample mixture. The user can calculate the retention factor (R_{f}) by measuring the migration distance of the chemical dye and the migration distance of the solvent front (which, in the case of a gasoline fuel, is the migration distance of the gasoline). R_{f} can be mathematically described by the following formula: retention factor (R_{f})=migration distance of the chemical dye/migration distance of solvent front. There is a correlation between the retention factor and the ethanol content in the sample mixture.

The chemical dye may be a colored dye to enable a user to see the migration front of the dye during and after chromatography. The chemical dye is a dye that has the requisite chemical and physical properties to enable the differentiation of fuel mixtures having different alcohol content in a chromatography procedure. The retention factor (R_{f}) values should be sensitive enough to the polarity of the alcohol/fuel mixtures. The retention factor (R_{f}) values should be sensitive enough to distinguish between fuel samples that contain 0%, 5%, 10%, 20% and higher than 20% alcohol.

In some embodiments, the chemical dye is a porphyrin dye. Porphyrins are a group of heterocyclic macrocycle organic compounds, composed of four interconnected modified pyrrole subunits. The porphyrin ring structure is aromatic. Porphyrin molecules typically have very intense absorption bands in the visible region and are usually colored.

Through research and experimentation, the inventors have chosen a porphyrin derivative, namely, 5-(4-hydroxyphenyl)-10,15,20-triphenyl porphine (Frontier Scientific, Item no: 500071386, Catalog No: M40874, CAS: 87345-22-0) as an example suitable dye for the test strip applications. This particular porphyrin is commercially available from Frontier Scientific, Newark, Del. This particular porphyrin can be dissolved in dichloromethane. Under room temperature, this porphyrin solution is usually dark pink/red colored. This particular porphyrin appears to work for the test strip applications because of the presence of the phenol group (i.e., the hydroxyphenyl group) which is attached to the porphyrin ring. This increased the compound's polarity and its ability to interact with the silica gel plate, yielding retention factor (R_{f}) values that differed with different ethanol concentrations in samples.

Similarly, another porphyrin derivative, namely the 5-(4-hydroxyphenyl)-10,15,20-tritolyl porphine derivative, which also has one phenol group, appears to yield retention factor (R_{f}) values that differed with ethanol concentrations in samples as well. In chromatography tests conducted by the inventors, the 5-(4-hydroxyphenyl)-10,15,20-tritolyl porphine yielded nearly identical results as 5-(4-hydroxyphenyl)-10,15,20-triphenyl porphine.

Other types of dyes can be used in a similar way, provided that their retention factor (R_{f}) values vary with ethanol content in the ethanol/gasoline mixtures. This can be empirically determined by performing the methods as described in the present disclosure using standards (i.e., mixtures containing a precisely known concentration of ethanol). If a dye's retention factor (R_{f}) values stay relatively constant at all concentrations of ethanol (especially in the 0% to 20% range) in the fuel sample, the dye is generally not suitable for the test strip applications.

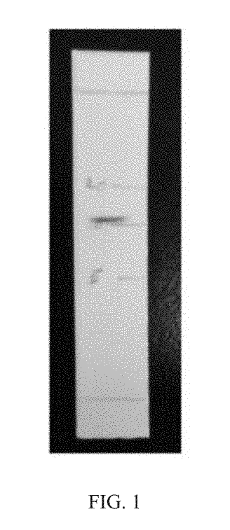

FIG. 1 shows an example test strip in the prototyping process. In FIG. 1, the test strip is a silica TLC strip. The strip has a length of 5 cm. The strip has a bottom baseline 0.5 cm from the bottom edge and a top baseline 0.5 cm from the top edge. The porphyrin derivative is deposited at the bottom baseline. The strip also has three marked lines (marked as “5”, “10”, and “20”) indicating the distance the porphyrin derivative would travel if the sample mixture contains 5%, 10% or 20% ethanol. These three lines can be termed 5%, 10% and 20% lines. FIG. 1 shows that when the strip is dipped into a 10% ethanol fuel sample, the porphyrin derivative migrated roughly to the 10% line, visually indicating to a user that the fuel sample contains 10% ethanol.

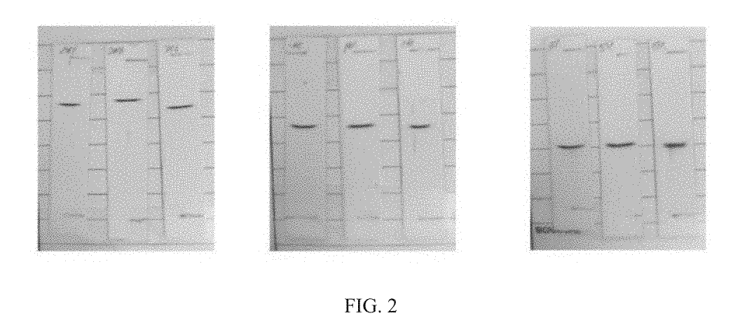

FIG. 2 shows that the measurements produced by the test strips are generally reproducible, when the same batch of prototype test strips were used. FIG. 2 has three panels. The left panel shows the results of three separate trials using a fuel sample containing 20% ethanol. The center panel shows the results of three separate trials using a fuel sample containing 10% ethanol. The right panel shows the results of three separate trials using a fuel sample containing 5% ethanol. FIG. 2 indicates that the porphyrin-based test strip can differentiate between fuel samples containing 5%, 10% and 20% ethanol, based on the migration distance of the porphyrin dye.

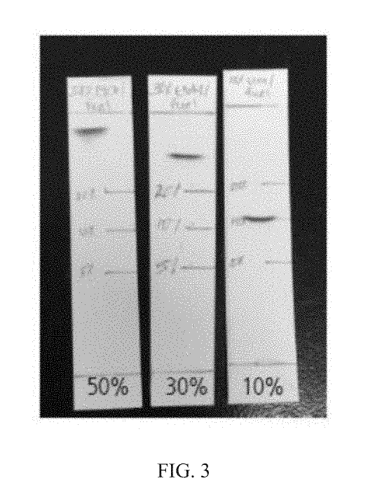

The porphyrin-based test strips can also be used to test fuel samples having an ethanol percentage greater than 20%. FIG. 3 shows three separate trials using fuel samples containing 50%, 30% and 10% ethanol. FIG. 3 indicates that the porphyrin-based test strip can differentiate between fuel samples containing 10%, 30% and 50% ethanol, based on the migration distance of the porphyrin dye.

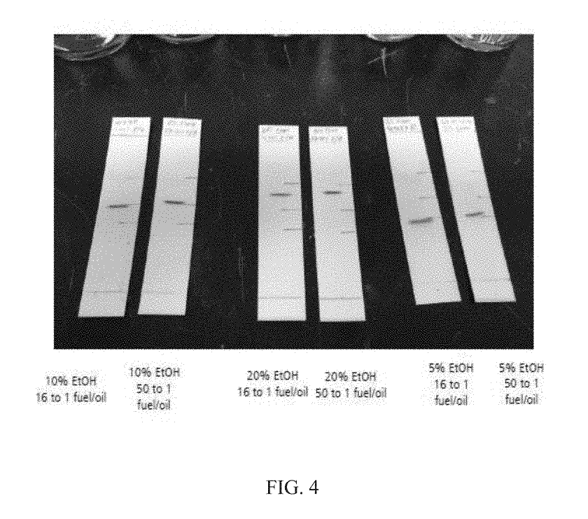

The porphyrin-based test strip can be used to determine the ethanol percentage in ethanol/fuel/oil mixtures. Fuel/oil mixtures are usually used in two-stroke engines and chainsaws. The working of a two-stroke engine is such that there is no space for lubrication; therefore, oil is mixed in with the fuel to provide lubrication. FIG. 4 shows that the porphyrin-based test strip can differentiate between fuel/oil samples (either 16 to 1 fuel/oil ratio or 50 to 1 fuel/oil ratio) containing 5%, 10% and 20% ethanol, based on the migration distance of the porphyrin dye. If needed, the marked lines for 5%, 10% and 20% ethanol on the test strip can be optimized for the oil containing samples. Such optimization may be done by performing tests using a set of standards (for example, 16 to 1 fuel/oil or 50 to 1 fuel/oil mixture standards containing known 5%, 10% and 20% ethanol content) to see where the porphyrin dye would migrate to. After the calibration tests, the test strips with the optimized marked lines can be used to measure fuel/oil samples of unknown ethanol percentage.

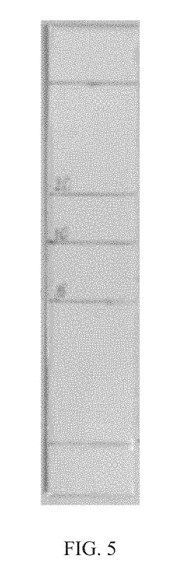

One aspect of the invention relates to methods of making test strips for measuring alcohol percentage in a liquid composition. In one example embodiment, the method comprises cutting silica TLC plates into strips (for example, 5 cm×1 cm size). The silica TLC plates may be commercially available (e.g., Merck KGaA item no. 105554). The method further comprises marking (e.g., in pencil) with a ruler a bottom baseline 0.5 cm from the bottom edge of the strip and a top baseline 0.5 cm from the top edge of the strip. Alternatively, the markings can be printed on the TLC plates using a printing machine (e.g., UV (ultraviolet) cured printing). Between the top baseline and the bottom baseline, additional marked lines can be marked or printed to correspond to different ethanol percentages. In a prototype example in FIG. 5, the 20, 10 and 5% lines are drawn in pencil. It should be noted that additional and/or alternative marked lines could be drawn or printed on the test strip (e.g., 15, 30, 40, 50 and 60% lines). It should also be noted that these marked lines may be calibrated and optimized by doing thin-layer chromatography using fuel or fuel/oil standards of known ethanol content.

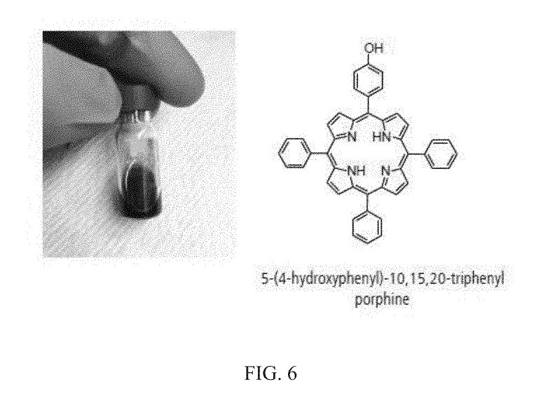

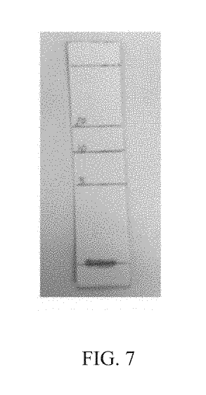

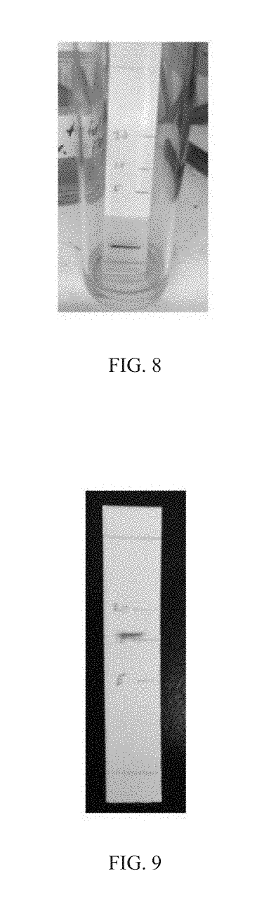

The method also comprises preparing a solution (1000 μM or 500 μM) of 5-(4-hydroxyphenyl)-10,15,20-triphenyl porphine (Frontier Scientific, item no: 500071386, CAS: 87345-22-0) in dichloromethane. The solution should be clear and dark pink/red colored. FIG. 6 shows a vial containing a porphyrin solution and the chemical structure of 5-(4-hydroxyphenyl)-10,15,20-triphenyl porphine. The method also comprises carefully spotting about 5 μL of porphyrin solution along the bottom baseline of the strip. The porphyrin solution should not be placed too close to the edge of the strip. The spotted line will change from red to brown as the dichloromethane evaporates. The test strip is now ready to be used. FIG. 7 shows an example test strip with porphyrin solution spotted on the bottom baseline.

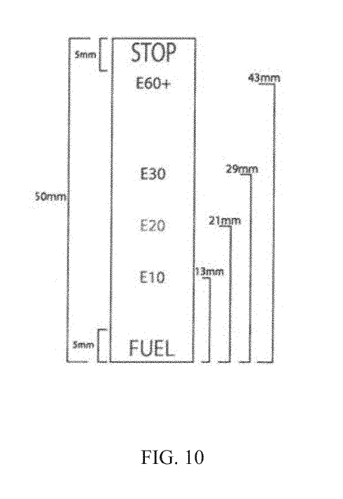

In another example embodiment which relates to a scaled-up production of these test strips, the method of making test strips for measuring alcohol percentage in a liquid composition starts with providing silica gel aluminum backed 20×20 cm TLC plates. UV cured ink is applied to print designs, marks, lines, and/or text on the plates. UV cured printing is a photochemical process in which high-intensity ultraviolet light is used to instantly cure or dry inks. UV cured printing is advantageous compared to some other conventional printing methods, because the ink dries or cures instantaneously and therefore does not interfere with the substrate of the TLC plates. The ink forms a separate layer on the substrate (e.g., silica gel) of the TLC plates and does not penetrate the substrate, so the ink will not change the behavior of the chromatography process. FIG. 10 shows an example of a test strip print design. In the FIG. 10 example, the texts include (from bottom to top) FUEL, E10, E20, E30, E60+ and STOP.

Four rows of 20 strips are printed on each plate; therefore, each single test strip has a dimension of 5 cm×1 cm. After printing, the plates are inspected for print defects and damage. Next, a 500 μM porphyrin solution diluted in dichloromethane is prepared. For each plate, the porphyrin solution will be striped along each row of strips approximately 5 mm above the bottom edge and will align directly above the “FUEL” text. Next, a clicker press with custom die blade is used to apply perforated cuts around each strip. These perforations allow for easy separation of a defined grouping of strips while keeping the plate intact during transit. An individual test strip can be obtained by separating along the perforations. The use of perforations is advantageous in that it allows the manufacturer to produce a sheet of connected test strips which can be separated with ease later by the end-consumers.

Although the silica gel aluminum TLC plates are mentioned as an example suitable TLC plate material for preparing the test strips, the present invention is not limited to silica gel aluminum TLC plates. Other suitable TLC plates can also be used. Although 5-(4-hydroxyphenyl)-10,15,20-triphenyl porphine solution is mentioned as an example suitable dye for preparing the test strip, the present invention is not limited to 5-(4-hydroxyphenyl)-10,15,20-triphenyl porphine. Other suitable chemical dyes can also be used.

If alternative TLC plates and/or alternative chemical dyes are used, the resulting test strips should undergo testing to calibrate the E0, E10, E20, E30, E60 lines on the test strip. Each new batch of test strips should also undergo testing to calibrate the E0, E10, E20, E30, E60 lines on the test strip. When testing a new batch of test strips, the test strips should be tested against a series of fuel sample standards that have a known percentage of alcohol, such as E0, E10, E20, E30, and E60. E0 is a fuel sample that does not contain any ethanol. For a lab testing, the inventors have used Chevron 94 gasoline as E0. Chevron 94 gasoline is an ethanol-free gasoline. To generate E10 ethanol/fuel mixture, the inventors have mixed 9 ml of Chevron 94 gasoline with 1 ml of ethanol to generate 10 ml of E10. To generate E20 ethanol/fuel mixture, the inventors have mixed 8 ml of Chevron 94 gasoline with 2 ml of ethanol. To generate E30 ethanol/fuel mixture, the inventors have mixed 7 ml of Chevron 94 gasoline with 3 ml of ethanol. To generate E60 ethanol/fuel mixture, the inventors have mixed 4 ml of Chevron 94 gasoline with 6 ml of ethanol. If a manufacturer wants to make test strips specifically for determining methanol (instead of ethanol) content in a fuel sample, the manufacturer can conduct calibration tests using a series of fuel sample standards that have a known percentage of methanol.

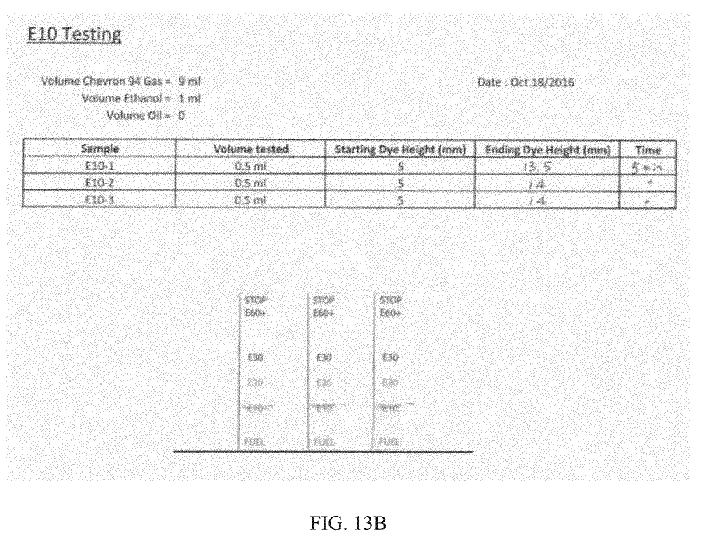

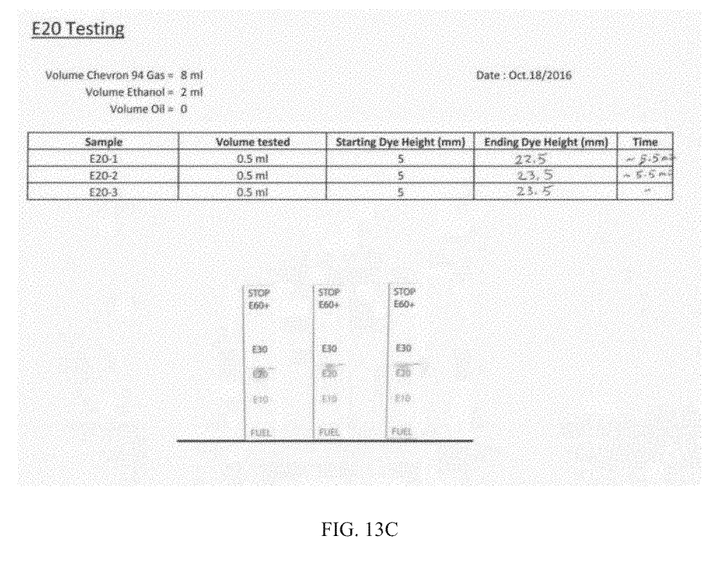

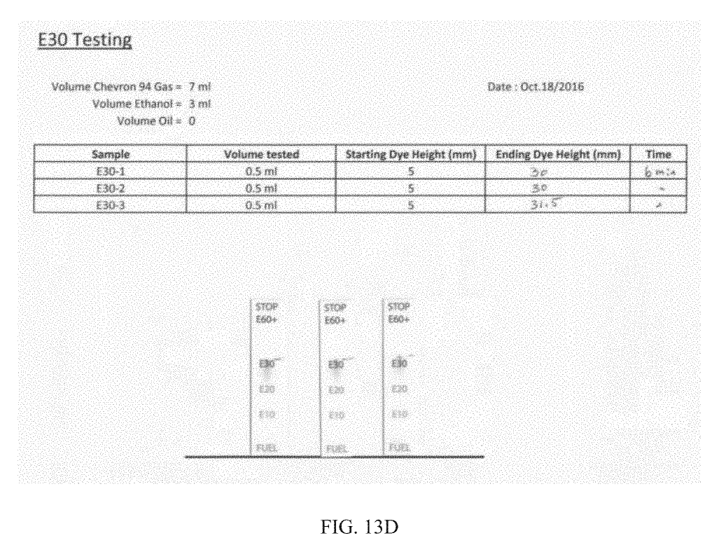

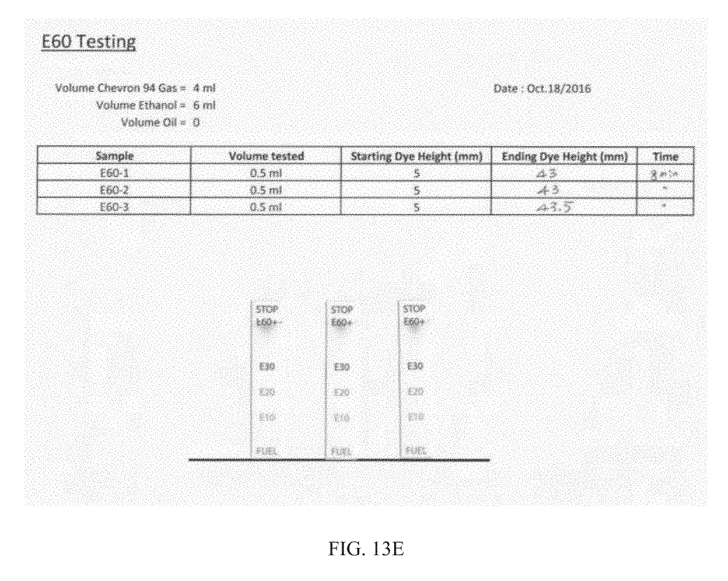

The inventors have tested a batch of test strips (made from silica gel aluminum TLC plates with 5-(4-hydroxyphenyl)-10,15,20-triphenyl porphine dye) in October 2016. These test strips have a dimension of 5 cm length by 1 cm width. The “STOP” line is at 5 mm from the top edge of the test strip. The starting dye line is at 5 mm from the bottom edge of the test strip. For sample E0, it took the gasoline sample 5 min to reach the “STOP” line and the ending dye height is 5 mm (i.e., unchanged). For sample E10, it took the gasoline sample 5 min to reach the “STOP” line and the ending dye height is 13.5 or 14 mm. For sample E20, it took the gasoline sample 5.5 min to reach the “STOP” line and the ending dye height is 22.5 or 23.5 mm. For sample E30, it took the gasoline sample 6 min to reach the “STOP” line and the ending dye height is 30 or 31.5 mm. For sample E60, it took the gasoline sample 8 min to reach the “STOP” line and the ending dye height is 43 or 43.5 mm. These calibration test results are shown in FIG. 13A through FIG. 13E. Based on the calibration results, the marked lines on the test strips can be confirmed or adjusted. The calibration tests can also be applied to calibrate samples that are ethanol/gasoline/oil mixtures. For such calibration tests, ethanol/gasoline/oil mixtures that contain a known percentage of ethanol (e.g., 0%, 10%, 20%, 30%, 60% etc.) can be prepared as standards.

The inventors have carried out additional tests using other types of samples, such as ethanol/TruFuel mixture, and ethanol/gasoline/oil mixtures. TruFuel 50:1 mix is an ethanol-free premixed 50:1 fuel/oil mixture for 2-cycle engines. It is commercially available in stores such as Home Depot or Lowe's. To generate E10 ethanol/TruFuel mixture, the inventors have mixed 9 ml of TruFuel 50:1 mix with 1 ml of ethanol to generate 10 ml of E10 ethanol/TruFuel mixture. To generate E20 ethanol/TruFuel mixture, the inventors have mixed 8 ml of TruFuel. 50:1 mix with 2 ml of ethanol. To generate E30 ethanol/TruFuel mixture, the inventors have mixed 7 ml of TruFuel 50:1 mix with 3 ml of ethanol. To generate E60 ethanol/TruFuel mixture, the inventors have mixed 4 ml of TruFuel 50:1 mix with 6 ml of ethanol. Ethanol/TruFuel mixtures are essentially ethanol/fuel/oil mixtures.

The inventors have also tested ethanol/fuel/oil mixtures which comprise Chevron 94 gasoline and Valvoline oil (50:1 Chevron/Valvoline) and ethanol or Chevron 94 gasoline and Briggs & Stratton oil (25:1 Chevron/Briggs) and ethanol. Some of the test results are presented in the Tables below.

TABLE 1
Test Results Using Samples That Contain 10% Ethanol

| Fuel | Ethanol % | Ending Dye Height (mm) |
|---|---|---|
| Chevron 94 | E10 | 13.5 |
| Chevron 94 | E10 | 14 |
| Chevron 94 | E10 | 13.5 |
| Chevron 94 | E10 | 13.5 |
| Chevron 94 | E10 | 13.5 |
| TruFuel Mix | E10 | 14 |
| TruFuel Mix | E10 | 15 |
| Trufuel Mix | E10 | 14 |
| 50:1 Chevron/Valvoline | E10 | 12 |
| 50:1 Chevron/Valvoline | E10 | 12 |
| 50:1 Chevron/Valvoline | E10 | 12 |
| 25:1 Chevron/Briggs | E10 | 12 |
| 25:1 Chevron/Briggs | E10 | 12.5 |
| 25:1 Chevron/Briggs | E10 | 12 |
| Average | | 13.1 |
| Standard Deviation | | 1.0 |

TABLE 2
Test Results Using Samples That Contain 20% Ethanol

| Fuel | Ethanol % | Ending Dye Height (mm) |
|---|---|---|
| Chevron 94 | E20 | 22 |
| Chevron 94 | E20 | 20 |
| Chevron 94 | E20 | 22 |
| Chevron 94 | E20 | 22 |
| Chevron 94 | E20 | 21 |
| TruFuel Mix | E20 | 23 |
| TruFuel Mix | E20 | 23 |
| TruFuel Mix | E20 | 24 |
| 50:1 Chevron/Valvoline | E20 | 19 |
| 50:1 Chevron/Valvoline | E20 | 18.5 |
| 50:1 Chevron/Valvoline | E20 | 19 |
| 25:1 Chevron/Briggs | E20 | 21 |
| 25:1 Chevron/Briggs | E20 | 20 |
| 25:1 Chevron/Briggs | E20 | 22.5 |
| Average | | 21.2 |
| Std. Dev. | | 1.7 |

TABLE 3
Test Results Using Samples That Contain 30% Ethanol

| Fuel | Ethanol % | Ending Dye Height (mm) |
|---|---|---|
| Chevron 94 | E30 | 30 |
| Chevron 94 | E30 | 32.5 |
| Chevron 94 | E30 | 32 |
| Chevron 94 | E30 | 30 |
| Chevron 94 | E30 | 31.5 |
| TruFuel Mix | E30 | 31.5 |
| TruFuel Mix | E30 | 30 |
| TruFuel Mix | E30 | 30 |
| 50:1 Chevron/Valvoline | E30 | 26.5 |
| 50:1 Chevron/Valvoline | E30 | 26.5 |
| 50:1 Chevron/Valvoline | E30 | 26.5 |
| 25:1 Chevron/Briggs | E30 | 29 |
| 25:1 Chevron/Briggs | E30 | 27 |
| 25:1 Chevron/Briggs | E30 | 28.5 |
| Average | | 29.4 |
| Std. Dev. | | 2.1 |

TABLE 4
Test Results Using Samples That Contain 60% Ethanol

| Fuel | Ethanol % | Ending Dye Height (mm) |
|---|---|---|
| Chevron 94 | E60 | 43 |
| Chevron 94 | E60 | 42 |
| Chevron 94 | E60 | 43 |
| Chevron 94 | E60 | 43 |
| Chevron 94 | E60 | 42 |
| TruFuel Mix | E60 | 44 |
| TruFuel Mix | E60 | 44 |
| TruFuel Mix | E60 | 44 |
| Average | | 43.1 |
| Std. Dev. | | 0.8 |

TABLE 5
Test Results Using Samples That Contain 85% Ethanol

| Fuel | Ethanol % | Ending Dye Height (mm) |
|---|---|---|
| Chevron 94 | E85 | 42 |
| Chevron 94 | E85 | 42 |
| Chevron 94 | E85 | 43 |
| Chevron 94 | E85 | 43 |
| Chevron 94 | E85 | 43 |
| Average | | 42.6 |
| Std. Dev. | | 0.5 |

One aspect of the invention relates to methods of using the test strips as described herein for measuring alcohol content in a liquid composition. In one example embodiment, the method comprises adding a liquid mixture (the ethanol content of which is unknown to a user) to a chamber, sealable glass vial or jar. The amount of the liquid mixture required for the test may be 0.5 ml or less. The user should ensure that the liquid mixture level is below the bottom baseline (where the dye is) of the strip. The user should ensure that the chamber is large enough to accommodate the test strip (e.g., the chamber should be >5 cm in height), though narrow enough to allow the test strip to lay upright on an angle.

The method also comprises placing the test strip gently into the chamber, closing the lid and allowing the solvent to travel to the top baseline of the test strip. The method also comprises removing the test strip from the chamber (when the solvent front has travelled to the top baseline of the test strip) and visually reading the result. If the chemical dye has traveled to the 5% line, that indicates that the sample contains approximately 5% ethanol. If the chemical dye has traveled to the 10% line, that indicates that the sample contains approximately 10% ethanol. If the chemical dye has traveled to the 20% line, that indicates that the sample contains approximately 20% ethanol. If the chemical dye has traveled to the 30% line, that indicates that the sample contains approximately 30% ethanol. And so on.

The test strips as described in the present disclosure are advantageous. They provide a simple, effective, and inexpensive tool to a user to rapidly and visually determine the ethanol content of a fuel or fuel/oil mixture. In many circumstances, the ethanol content of a fuel or fuel/oil mixture is unknown to a user, and the test strips enable the user to do a quick test to find out the approximate content of ethanol in the sample. The test strip is also useful for a user who simply wants to find out whether ethanol is present in a sample. The test strip can provide the “Yes” or “No” answer. The test strips are light and portable. The user can use the test strip to determine the ethanol content of a fuel or fuel/oil mixture in diverse situation (e.g., at home, in a garage or outdoors). The test strips can be used in a field test. The test requires a very small amount of the sample (e.g., 0.5 ml). This is much less than a conventional test conducted using a graduated cylinder which requires at least 20-100 ml of fuel sample.

One aspect of the invention relates to a kit wherein the kit comprises a plurality of ready-to-use test strips as described in the present disclosure and an instruction sheet explaining to a user how to use the test strips to determine the ethanol content of a fuel or fuel/oil mixture. The kit may be packaged in a case or container. The kit may be sold as a unit to users in a retail store or through an online store.

In an example embodiment, the kit comprises a plurality of ready-to-use test strips as described in the present disclosure, a plurality of test result labels, a pipette, a fuel test vial, a small tool (such as tweezers for handling and/or picking up the test strip), and a printed package insert which provides information and instructions to the user.

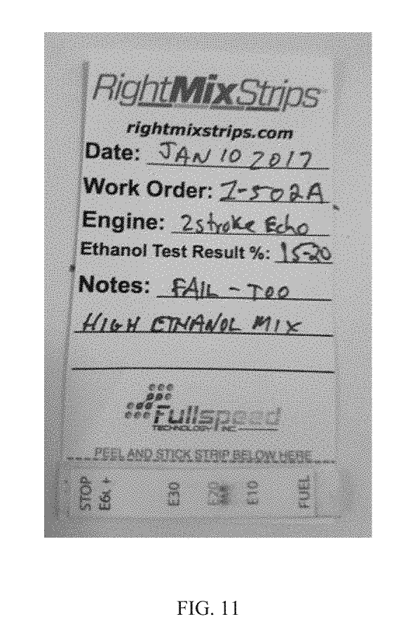

The test result label is a useful component of the kit. The test result label is a printed label that allows the person performing the test to attach the test strip and present the test strip result and information to a customer who has requested the test. The test result label allows the person performing the test to write information such as date, work order number, engine type, test result (such as percentage of ethanol content in the fuel), and notes on the label. The test result label also allows the actual test strip to be attached and presented in combination with the information. FIG. 11 shows an example test result label.

The presence of the test result label is advantageous in that it allows a business (such as a shop or gas station) to provide a diagnostic fuel test service for a paying customer and then presenting the test results to the customer. The customer would pay the business for performing the test and presenting the test results on the test result label.

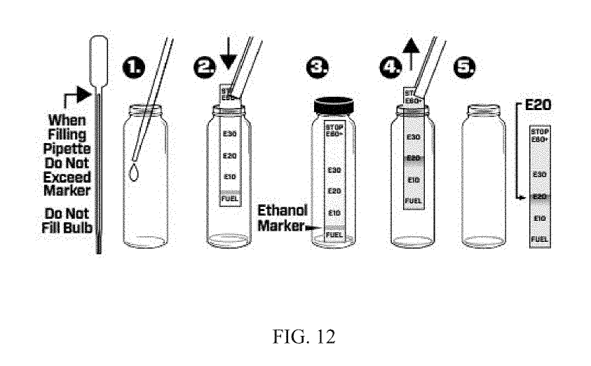

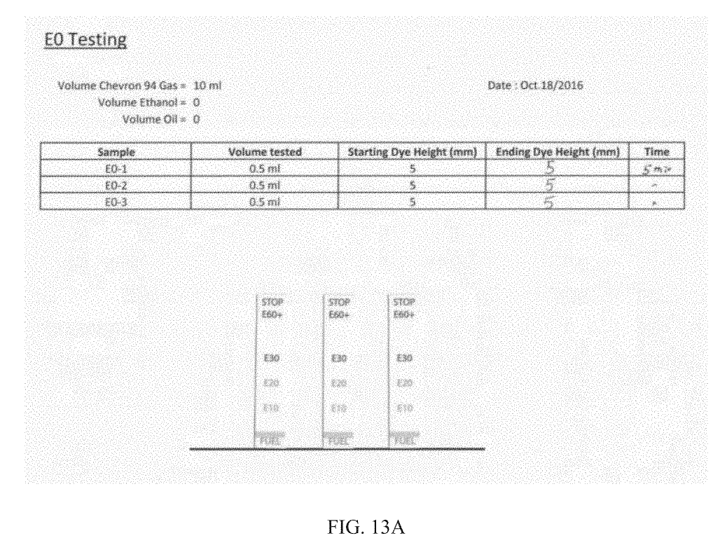

FIG. 12 is a pictorial that illustrates the steps of using the test strip provided in a kit to determine the ethanol content in a fuel sample. In step 1, a user uses a pipette to draw a small volume of fuel sample (e.g., 0.5 ml of gasoline) into the pipette, up to a top marker line under the bulb of the pipette and then transfers the fuel sample into the fuel test vial. In step 2, the user uses tweezers to gently place a test strip into fuel test vial, with the “FUEL” end pointing towards the fuel sample. In step 3, the user screws a cap on the fuel test vial, without lifting, shaking or moving the fuel test vial. The user waits a few minutes until the fuel sample travels up the test strip, from bottom “FUEL” position to “STOP” position, but not to the very top end of the test strip. In step 4, the user immediately removes the test strip from the fuel test vial and let it air dry. Optionally, the user can attach the test strip to a test result label. This is primarily for shop use. In step 5, the user determines the ethanol content in the fuel sample based on the migration front of the color dye. In the FIG. 12 example, if the ethanol marker (i.e., the color dye) has not moved on the test strip from the original position, that indicates that the fuel sample is ethanol free. If the ethanol marker has moved, the user can find the approximate ethanol content in the fuel sample by comparing the position of the ethanol marker (i.e., the color dye) with the lines and/or text printed on the test strip. Before the test strips and the kits are sold to consumers, the lines and/or text printed on the test strip should have been calibrated using fuel standards by the manufacturer.

It is understood that the examples in the foregoing disclosure in no way serve to limit the scope of this invention, but rather are presented for illustrative purposes. As will be apparent to those skilled in the art in the light of the foregoing disclosure, many alterations and modifications are possible in the practice of this invention without departing from the scope thereof.

1. A test strip for determining the concentration of a component in a liquid composition, the test strip comprising a carrier sheet coated with a layer of an adsorbent, and a chemical dye deposited on the test strip near but spaced apart from a bottom edge of the test strip.

2. The test strip according to claim 1, wherein the carrier sheet coated with the adsorbent layer is a thin-layer chromatography (TLC) plate.

3. The test strip according to claim 2, wherein the retention factor of the chemical dye is dependent on or has a correlation to the concentration of said component in said liquid composition, thereby a user can determine the concentration of said component based on the migration distance of the chemical dye on the test strip in a chromatography process.

4. The test strip according to claim 3, wherein said liquid composition is an alcohol/fuel mixture or alcohol/fuel/oil mixture and the test strip is for determining the alcohol concentration in the mixture.

5. The test strip according to claim 4, wherein the alcohol is methanol, ethanol, propanol, or butanol.

6. The test strip according to claims wherein the alcohol is ethanol.

7. The test strip according to claim 6, wherein the test strip is for determining the concentration of ethanol in said liquid composition, and the ethanol concentration is between 0 to 99%.

8. The test strip according to claim 7, wherein the ethanol concentration is between 0 to 60%.

9. The test strip according to claim 8, wherein the test strip comprises a bottom baseline on which the chemical dye is deposited, and the bottom baseline is spaced apart from the bottom edge of the test strip.

10. The test strip according to claim 9, wherein the test strip comprises a top baseline near a top edge of the test strip which serves as an indicator to a user that the chromatography process should be stopped when a solvent front reaches the top baseline.

11. The test strip according to claim 9, wherein the test strip comprises one or more indicator lines or markings or text elements, and a user can determine the concentration of said component by comparing the migration front of the chemical dye with said one or more indicator lines or markings or text elements.

12. The test strip according to claim 11, wherein the chemical dye is a porphyrin derivative.

13. The test strip according to claim 12, wherein the chemical dye is 5-(4-hydroxyphenyl)-10,15,20-triphenyl porphine.

14. A test kit for determining the concentration of a component in a liquid composition, the test kit comprising: a plurality of the test strips as defined in claim 1, a plurality of test result labels, a pipette, a fuel test vial, a tweezer-type tool for handling or picking up the test strips, and a printed package insert which provides information and instructions to test kit users.

15. A method of making a test strip for determining the concentration of a component in a liquid composition, the method comprising: (1) providing thin-layer chromatography (TLC) plates and cutting the TLC plates into strips, (2) marking a top baseline near a top edge of the strip and a bottom baseline near a bottom edge of the strip, (3) marking one or more indicator lines or text elements on the strip, (4) dissolving a chemical dye in a solvent to form a chemical dye solution, (5) spotting an amount of the chemical dye solution along the bottom baseline on the strip, and (6) allowing the solvent to evaporate from the strip.

16. The method according to aspect 15, wherein the chemical dye is 5-(4-hydroxyphenyl)-10,15,20-triphenyl porphine and the solvent is dichloromethane,

17. A method for determining the concentration of a component in a sample mixture, the method comprising: (1) adding the sample mixture into a chamber, (2) placing a test strip as defined in claim 1 into the chamber, ensuring that the sample mixture is below the bottom baseline of the test strip, (3) covering the chamber with a lid and allowing the sample mixture to migrate to the top baseline of the test strip, (4) when the front of the sample mixture has reached the top baseline of the test strip, removing the test strip from the chamber, and (5) visually inspecting the migration front of the chemical dye to determine the concentration of the component in the sample mixture.

18. The method according to claim 17, wherein the component is an alcohol and the sample mixture is an alcohol/fuel mixture or alcohol/fuel/oil mixture.

19. The method according to claim 18, wherein the alcohol is ethanol.

20. The method according to claim 18, wherein the alcohol is methanol.
